(19) United States
(12) Patent Application Publication
(10) Pub. No.: US 2012/0271293 A1
(43) Pub. Date: Oct. 25, 2012

(54) Method for targeted local heat ablation using nanoparticles

(76) Inventors: Ze'ev R. Abrams, Berkeley, CA (US); Lawrence M. Abrams, Englewood, NJ (US)

(21) Appl. No.: 13/200,041

(22) Filed: Sep. 15, 2011

Related U.S. Application Data
(60) Provisional application No. 61/478,512, filed on Apr. 24, 2011.

(51) Int. Cl. A61B 18/04

(52) U.S. Cl. 606/28

(57) ABSTRACT

This invention relates to the targeting of specific tissue for destruction or modification using electromagnetic radiation coupled with nanoparticles to locally apply heat to the targeted tissue by concentrating the energy in a temporary or permanently placed medium. In general, this invention addresses the need to ablate, i.e., to reduce, eliminate, or to impede growth in specific tissue; and, to do so in a highly targeted and completely controllable implementation. Specific examples are described, focusing on, but not limited to, the retardation, reduction, and/or elimination of obstructing material and tissue in vascular stents and gastro-esophageal valves. For illustrative purposes, other examples are mentioned. Ablation is induced by the nano-plasmonic effect in metallic-based nanoparticles including, but not limited to, gold and gold coated nanoparticles; a wide variety of alternate materials are equally suitable.

### BACKGROUND OF THE INVENTION

1. Field of the Invention

The present invention describes devices for treating various dysfunctional bodily functions through the use of targeted ablation using a combination of electromagnetic radiation and nanoparticles. The nanoparticles are embedded upon various embodiments of devices including stents and catheters, which are then implanted or temporarily placed within the body.

2. Description of the Related Art

Energy induced ablation is a well described and active field in procedural medical practice. All applications share the common objective of reducing, eliminating, or otherwise modifying live tissue or, in modifying the consequences of pathological processes, such as inflammation, or, abnormal or detrimental tissue growth. Most early ablation applications, many of which are still in use, have directly applied local heat to targeted tissue. Later ablation techniques have evolved to include energy transfer using radio-frequency electrodes to generate heat directly. Still later, highly focused optical ablative energy, such as in lasers, were developed. All of these methods share the common deficiency of poor control over the extent and intensity of the local tissue destruction because they are difficult to control, limit and focus, and cause collateral damage to surrounding tissue. In each case, the techniques' deficiency is analogous to the comparison of using a cannon where a scalpel would serve better. One object of the present invention is to control and limit the energy and focus of the ablation.

Clinically important areas in which ablation is utilized include Gastro-intestinal, Urological, and Cardiac. Numerous other medical regions are being treated by suitable ablative techniques. All previous clinical applications have had limited success in targeting and limiting the ablation energy, consequently, an ideal methodology should have the fewest long term negative effects. Long term negative effects include excessive acute damage or negative chronic changes induced by excessive or insufficient energy. Included in the long term positive benefits should be the ability to repeat the procedure as often as is necessary, with minimal indirect trauma due to the invasive nature of internal ablation procedure, as well as a mechanism by which fine-tuning of the ablative process can be achieved.

With these limitations in place, new research has been focused on creating materials and methods to locally heat tissue using relatively noninvasive methods. Amongst these methods, the field of Plasmonic Photo Thermal Therapy (PPTT) has gained ground due to its ability to achieve localized heating in the required temperature range, while being limited to a spatially confined region. PPTT is based on the effect of light upon small nanoparticles such that light impinging upon the nanoparticles activates a resonant oscillation of the electrons in the nanoparticles (plasmons), which, due to their small size, then dissipate the energy as heat into the local environment. The nanoparticle's plasmonic resonance can be tuned to react to specific wavelengths of electromagnetic radiation, as a function of the size, material and coatings used. In particular, gold and silver nanoparticles will typically have resonance peaks in the visible spectrum, whereas gold-coated silicon nanoparticles can be tuned to resonate when excited by Near Infra-Red (NIR) light. The tunability of these nanoparticles allows one to selectively control the wavelength of electromagnetic radiation required to cause these nanoparticles to resonate. Furthermore, these nanoparticles remain both physically and biologically inert until their excitation by their tuned resonant wavelength. The temperatures reached using PPTT are a function of the concentration of nanoparticles used, and can easily reach local temperature increases of tens of degrees sufficient to cause the thermal ablation of adjacent cells.

PPTT has thus far been primarily focused on the treatment of cancer, with nanoparticles injected into cancerous growths and excited by light, causing the thermal ablation of the cancerous cells. This process has been demonstrated in U.S. Pat. No. 6,530,944 for suspensions of colloidal nanoparticles, with or without surfactants, and has been shown to be a viable method for locally ablating tissue. The nanoparticles used for these applications are typically injected into the tissue, and can be chemically labeled with selective antibodies so as to selectively bind to cancer cells, thereby adding the capability to spatially select cells and limit collateral cell ablation. The nanoparticles injected into the body are eventually cleared out through filtration in the liver. The nanoparticles remain inert in both the biological toxicity sense, as well as the optically activated sense, as they cannot be excited within the tissue by any other method other than their specific resonance wavelength. The methods described previously using nanoparticles for diagnostic treatment have been limited to cancer treatment or tissue repair, with the same physical properties of the PPTT used for the ablation of cells. However, the use of nanoparticles and PPTT is here used for localized tissue ablation, with the nanoparticles embedded within the device described here, and therefore constitute an entirely different embodiment of the PPTT concept. Furthermore, the focus of this invention lies in its physical embodiment and use, and is not limited to the specific type of nanoparticles used for the PPTT process, as opposed to the previous methods, which were limited to therapeutic and diagnostic usage of suspended nanoparticles injected into target tissue, or used for non-PPTT methods.

The first embodiment of this invention will focus its examples on vascular and Cardio-vascular applications, especially in the use of ablation in stent related complications. While stent development and research has centered on Cardio-vascular applications, it is by no means limited to Cardio-vascular structures. In fact, pathologically abnormal conduits anywhere in the body are continuing to evolve stent related applications, and the described techniques are intended to apply to all stent applications.

Stent use in Cardio-vascular applications first evolved as a means of providing a kind of scaffolding to damaged vascular structures, usually narrowed and diseased coronary artery conduits, that had been dilated, usually with a balloon, a procedure known as Angioplasty, and which subsequently depended upon the scaffolding to prevent secondary collapse, or to prevent reparative tissue overgrowth. These early stents, now known as Bare Metal Stents (BMS), successfully prevented the immediate and early collapse of the successfully dilated vessel. However, they failed to always prevent the intermediate (around 3 months) and long term consequence of new reactive vascular tissue growth that would grow into the stent lumen, leading to significant, and sometimes complete, narrowing or occlusion. There is extensive recorded morbidity and mortality associated with this secondary growth within and in the immediate proximity to BMS.

Consequently, a new type of stent, which is coated with an anti-metabolic drug, the Drug Eluting Stent (DES), was developed, to specifically address this problem. The DES has successfully reduced or eliminated the problem of secondary tissue growth, but has done so with a cost. The chemicals used are so efficient that they destroy all tissue in the immediate vicinity of the stent, and, the chemicals often diffuse a short distance to damage adjacent tissue; the chemicals continue to do so until the drug is absorbed and eliminated by the body. The dose is fixed, by default, at the factory specification for the amount of drug applied to the stent. The duration, i.e., the on-off switch, is predefined as the insertion time (on) and the eventual drug elimination (off). In short, the DES is a “one size fits all” solution that is applied more like a “cannon shot”, or, as it has often been described, as a multi-month, around 6 months, long-slow burn, rather than as an individually tailored precision therapy.

The negative consequences of the DES approach are now becoming evident. The vascular wall, as well as the nearby tissue that has been damaged or destroyed by the chemicals in the DES, often form scar tissue. The scar tissue may fail to form the protective epithelial covering known to be essential to vascular function. Since the stent, usually a BMS, that forms the substrate for the chemical surface coating of the DES, is a foreign object, the uncovered metal struts may adversely interact with blood components. One such interaction is clot formation caused by turbulent flow locally occurring in the vicinity of the metal struts. The turbulence stimulates platelets to form clots. Normal epithelium impedes this process; if the stent surfaces were covered by normal endothelium, thrombus and clot would be impeded. The DES induced scarred vessel surface, a direct result of over dosing the chemical ablation, cannot form the required properly functioning normal epithelium because the vessel is too badly damaged to form normal tissue, and thus forms scar. It is the absence of this re-established normal epithelium that typically beneficially prevents unwanted inflammatory occlusive in-growth into the lumen of the stent, but comes with the disadvantage of failing to prevent clot, i.e., thrombus, formation.

Numerous reports of sudden late thrombus formation in vessels in which a DES has been placed have been published. This acute thrombus sometimes leads to rapid fatal consequences, but at the least, can cause ischemia or frank myocardial infarction. Consequently, the statistical morbidity and mortality benefits gained with use of DES, which prevents the slow secondary occlusion of the vessel that is seen with the BMS, is countered in a DES with and equally potentially disastrous complication. When a DES is the choice of stent, the benefit of reduced slow occlusion is countered by the equally serious occurrence of statistically significant sudden vessel occlusion that may follow a DES insertion. While the medical literature is still unclear about the relative merits of the two approaches, the market has clearly spoken; DES sales are far below company expectations, and the prevailing opinion is that the market hesitance is directly related to the devastating potential thrombus formation as a consequence of the DES insertion. When patients and cardiologists, knowing the potential pros and cons of the DES, do decide to opt for a DES, that decision now is made with the mandatory unending requirement for the patient to take dangerous anti-platelet therapy for the rest of his or her life in an attempt, though not always successful, to prevent the formation of this devastating acute thrombotic complication.

It is an object of the first described embodiment of this invention to provide a more controlled, yet equally effective, tissue ablation approach that will prevent and will also treat tissue encroachment within stents by providing only the therapeutically required ablation, while still allowing a normal endothelial layer to form over the stent. Equally, it is an object of this invention to offer a technique so robust that it can be repeated as often as is necessary once the stent is in place.

A second embodiment of this invention involves the more general use of ablation techniques for vascular, gastro-esophageal and other uses. Ablation techniques are widely applied in applications where stents are not applicable, or where tissue encroachment is not an issue. Examples include ablation of conduction pathways in proximity to the surface of cardiac tissue or within the walls of vessels associated with the heart, such as the pulmonary veins. These ablation techniques are performed in an attempt, supported by extensive clinical experience, to correct abnormalities in tissue or nerve conduction in conduction tissue, which may lead to dangerous or disabling cardiac rhythm disturbances, for example, Atrial Fibrillation. Atrial Fibrillation is often caused by an abnormal origin of a conducting signal in or near a cardiac chamber, usually on the left side of the heart, or, in a vessel leading to the left side of the heart, such as a pulmonary vein. Ablation of these conduction pathways can successfully disrupt these pathological rhythm pathways, and allow the normal conduction pathways to restore normal heart beat. Currently used ablation technologies include direct application of heat, focused radio-frequency, laser or ultrasonic, and, in some cases, actual surgical ligation of the targeted tissue. These techniques have the associated problems of ineffective control over the amplitude, duration, and extensiveness of the ablation, causing collateral damage to nearby tissue. Furthermore, they typically can only be applied once, with additional procedures involving considerably more possible complications.

The most common atrial arrhythmia is atrial fibrillation. Other “tachy-arrhythmias” also occur and most have the common denominator of originating in tissue adjacent to either, most frequently, the left atrium or, less frequently, the right atrium. Long thought to be benign at best, or an “irritant” at worst, recent evidence suggests that these arrhythmias significantly shorten life and cause degraded life quality. Such arrhythmias also result in major health care costs and loss of economic productivity for those who would otherwise choose to work. Treatment of Atrial Fibrillation alone is one of the healthcare system's and Medicare's major cost items.

While physicians have preferred to initially opt for pharmacologic therapy, the failure of the drug treatment often leads to an invasive catheter based therapy targeting the arrhythmia source sites for tissue ablation. Ablation methods most commonly employ energy delivery, such as heat or Radio Frequency (RF), but also may employ a freezing, often referred to as a “cold” or cryo techniques. Importantly, these methods almost universally deliver excess heat, energy or cold to the targeted area. The basis for this excess delivery is justified by a belief that the clinician will get only “one shot” at solving the problem, either because of patient preference or due to potential complications from the procedure. The excess heat or cold delivery is also a result of the physical limitations of the catheters themselves.

In practice, direct RF delivery, resistance heated energy delivery, or cryo cooling delivery requires a catheter that is large enough to house the wire, fluid transmission, and/or other materials needed to deliver significant energy. Such a catheter cannot be very small and would need to be relatively rigid. Both of these physical limitations significantly limit the maneuverability of the catheter, especially in small chambers such as the left atrium, and particularly when there are numerous areas within that small chamber that must be precisely targeted.

For atrial arrhythmias, the targets are usually the pulmonary veins, conduits that carry oxygenated blood from the lung alveoli to the left atrium of the heart so that the heart can pump this oxygenated blood to the rest of the body. It is well established that many atrial arrhythmias, including atrial fibrillatin, originate most commonly in the pulmonary veins, usually at a location just before the pulmonary vein enters the left atrium.

Historically, the distal or exiting segments of pulmonary veins were targeted for ablation using precise ablation directed at just the correct tissue area. These areas can be very accurately identified by catheter based electrophysical mapping methods. However, focused and localized ablation to the precise origin of the arrhythmia failed, as the source site simply moved to a nearby undamaged area. Consequently, the entire circumference of the distal pulmonary vein was targeted. Such a technique is difficult since the catheter is smaller than the circumference of the distal pulmonary vein. Significant manipulation coupled with excess energy delivery was the solution, since precise manipulation of the catheter alone to treat the entire circumference without leaving open areas was nearly impossible and was therefore by itself insufficient. The excess energy allowed tissue destruction to reach areas near to but not touching the catheter.

Consequently, there were severe problems with these techniques. Delivering energy to all of the tissue circumferentially often resulted in a miss of the targeted area, and therefore such technique is at best a “hit or miss” approach. As a result, a “miss” could mean requiring a repeat procedure, with no guarantee that there would be a precise “hit” to the target area. Importantly, each treatment caused significant scarring of the tissue forming the pulmonary vein wall. Because scars contract, the scarring of the pulmonary vein wall has been a significant problem. Any contraction sufficient to restrict or even occlude flow through the pulmonary vein caused major complications, sometimes fatal. Failure to stop the arrhythmia with a first treatment episode led to understandable extreme caution when considering a repeat procedure, which also was a problem in that the initial condition was not corrected.

Other current approaches to tissue destruction ablation now avoid targeting the distal lumen of the pulmonary veins. Instead, ablation is performed at the exit point of each vein into the left atrium, but on the atrial wall, not in the lumen of the vessel. This is analogous to drawing a circle around an opening. This process is cumbersome, time consuming and less than a completely successful method. Similar to the other prior methods, excess energy is applied to better achieve complete and continuous tissue ablation of contacted and nearby tissue, since it nearly impossible with current catheters to “touch” all parts of the circle. As before, failure to “close the circle” leads to a failure of the procedure itself. As before, the excess scaring, while less likely to cause vessel occlusion, still precludes or restricts repeat treatments.

Another disorder for which ablation has been used as treatment is Barrett's Esophagus, which is a disorder of the lower esophagus, near the connection with the stomach (i.e., the Gastro-Esophageal Junction). This disorder is caused by stomach acid refluxing into the esophagus and is recognized on tissue microscopy analysis as gastric tissue replacing normal esophageal tissue. While not cancerous, this normal stomach mucosa tissue in this abnormal location can and sometimes does undergo change, known as Dysplasia. With continuing further change this Dyslpastic tissue can become cancerous. Because of the known cancer potential of Barrett's Esophagus, physicians often opt to destroy this Dysplastic tissue by ablation.

Existing ablation methods, as discussed above, include RF, heat, and cryo (i.e., cold) tissue destructive methods. As discussed above, these methods have significant problems and inefficiencies, cause excessive tissue injury and subsequent scaring. Ablation remains the most successful, minimally invasive technique for destroying such Dysplastic tissue.

Another disorder for which ablation has been used as treatment is asthma, which is rapidly increasing in prevalence worldwide in people of all ages. Asthmatics, on tissue microscopic analysis, have airway or bronchial passage ways that show thickened lining tissue, known as mucosa. Steroids and bronchial dilator drugs have become the mainstay in Asthma therapy, and with great success. However, some patients remain refractory to pharmacological treatment.

Recently, bronchial ablation has been shown to improve patients with asthma that is refractory to pharmacological therapy. Unfortunately, the ablation methods are the known methods of energy delivery, including RF, Heat, and Cryo cold techniques, as discussed above. However, damaging bronchial tissue with excessive energy and its resultant scaring has been shown to sometimes significantly worsen these patients, and quite possibly fatally.

Therefore, needed is a gentle heat application methodology that could be repeatedly administered with known tissue reducing effects. Such a method would be a significant improvement over the existing known methods, leading to an effective adjunct to current pharmacological therapy.

Still another problem for which ablation methods are used as treatment involves the sinus cavities, which communicate with the airway passages via small orifices. Infection, inflammation, allergy, and congenital factors can lead to one or more of the sinus orifices narrowing, stenosis, or closure. Stenting has recently been introduced as a method of opening and maintaining patency of these orifices. However, stents may close, and be difficult to replace. Even if replaceable, there are significant limitations on the number of replacements possible. Chronic closures of the sinus cavities would then require surgery to re-establish open passageways and orifices.

An effective ablation technique can be used to treat sinus outlet, narrow orifice disease, possibly reducing the need for a stent, and treat stent closure without requiring replacement of the stent. Thus, many more patients may be treated by means less invasive than surgery.

Yet another disorder for which ablation techniques serve as treatments include narrowing of passageways in the gastro-intestinal (GI) tract, which often occurs in the pediatric and neonatal population, as well as in adults with various diseases, and . The outlet of the stomach into the intestines, and the ducts draining the Gall Bladder are examples. While known ablation techniques may be employed, positioning can be difficult and can often lead to similar problems as discuss earlier.

Similarly, the passageways from the kidney to the bladder, including the critical junction areas, and the passageway from the bladder via the urethra, including the bladder junction, the prostatic passage, and the urethra exit points in men, women, and children, are all subject to disease induced or congenital narrowing. Also, in women with diseases of the fallopian tubes, the passageways from the ovary to the uterus may have either disease induced narrowing or may be congenitally narrowed leading to reduced fertility or infertility. Likewise, women who have tube closure for birth control, and who later wish to reverse this decision, may have difficulty keeping their fallopian tubes open after surgical reversal. Women with excessive uterine bleeding may have ablation procedures to reduce the tissue thickness of their endometrial mucosa, as a means of bleeding control. Heat and other tissue destroying methods are currently employed. While such known ablation techniques may be employed, they can often lead to similar problems as discuss earlier.

Consequently, there is a need for a gentle, targeted heat application methodology that could be repeatedly administered with known tissue reducing effects. Such a method would be a significant improvement over the existing known methods.

The present invention will add a new modality for achieving results that are equivalent to or superior to current technologies. The described technique will offer the precision of surgical ligation with the benefit of minimal tissue destruction and the opportunity to repeat the procedure as often as is necessary. Current techniques, by their nature can be utilized only for a very limited number of times, since the scarring that results from the extensive tissue destruction can lead to very serious outcomes. As an example, targeted pulmonary veins, which are the conduits of blood entering the left heart from the lungs, may chronically scar after ablation and subsequently obstruct blood flow into the heart, as a direct result of overly extensive tissue damage secondary to currently applied ablation techniques used to treat Atrial Fibrillation.

Another example of a currently applied ablation procedure that would benefit from this invention involves treating conditions of the esophagus, usually near the junction with the stomach, the Gastro-Esophageal junction. Ablation techniques for the treatment of Gastric Reflux (GERD), or therapies designed to treat esophageal motility problems, focusing on the lower esophageal area. Excessive scarring here is catastrophic, potentially leading to complete obstruction of oral food passage from the Esophagus into the stomach. Precision, targeted, surgically equivalent methodological techniques, that can be repeated as often as is necessary, would be a real advance in this area.

There are other areas, too numerous to list here, that would benefit by this technique. Hence, this invention describes a generally applicable technique that can find useful application in any medical situation requiring controlled precise ablation.

### SUMMARY OF THE INVENTION

To successfully apply the principles of this invention, two conditions are preferably desired. First, there should be a locally applied, installed or externally directed electromagnetic energy source, delivered only when desired. Second, there should be present, either temporarily or permanently, a structure containing nanoparticles embedded within, or strongly bound to the surface, that is preferentially adjacent to the local therapeutic site. Thus, the preferred sequence is: insertion of the device within the required region to be treated; and delivery of electromagnetic energy locally to the device, resulting in heat concentrated directly on the target tissue or area. This dose can then be controlled via feedback control to supply the desired ablation dosage.

The electromagnetic radiation to be applied to the device is dependent upon the exact type of nanoparticles utilized, and is not limited to any distinct segment of the spectrum. These nanoparticles can include, but are not limited to, spherical and non-spherical metallic and non-metallic core-shell structures as well as nanoparticles that are fabricated directly onto the device, or pre-fabricated in a solution state. The wavelength resonances of these nanoparticles can be in the visible spectrum (i.e., 380-700 nanometers (nm)) and NIR spectrum (i.e., 700-2000 nm), and can include multi-resonant nanoparticles. A preferred wavelength for excitation of nanoparticles is in the NIR regime due to the low absorption of NIR light in human blood and tissue, thus maximizing the transmission of the excitation signal. The size of the nanoparticles is likewise independent, and should preferably have total dimensions in the range of 1-1000 nanometers.

In a preferred embodiment relating to stents, BMS (or DES) are further modified with the additional embedding (described below) of nanoparticles on the entire surface of the stent. These stents remain thermally inert until excited with the nanoparticle's resonance wavelength from a well controlled source. The electromagnetic source can be externally applied, such as to the chest wall from a source near to the skin, or it can be internally applied, such as from an intra-vascular source. This source might be a filtered lamp, LED or laser, with the wavelength defined by the nanoparticles utilized. Exciting the nanoparticles attached to the stent will then cause them to resonate, generating localized heat which is then transferred to the nearby tissue, and thereby engaging the PPTT. The intensity and duration (pulsed or continuous) of the irradiating light source determines the temperature that the nanoparticles reach and the ensuing PPTT. This irradiation is externally controlled, and typically will last a few seconds to a few minutes.

The PPTT response of the nanoparticle coated stent is to thermally ablate only those cells encroaching into the stent, or the ablation of platelets, the actual clot or the old clot adhering to the stent, thereby preventing clotting of the vessel. This localized ablation would have minimal effect on the intima layer of the vessels, thereby allowing the vessels to heal and allowing normal endotheliazation. For a DES, unwanted hyper-endotheliazation can be removed. The energy source can be placed using conventional and readily available catheters currently used for medical intra-vascular diagnostic and therapeutic procedures. This allows the PPTT to be implemented at later points after the surgical implant of the stent via a comparatively simple catheter procedure, and refraining from a complete removal of the stent. Internally implanted light sources, such as micro-LEDs, can be used as well, with the internal light source activating the PPTT with an external command. Finally, an external electromagnetic source can be used to excite the nanoparticles resonance by either an external source, or one inserted internally via non-vascular systems, such as in esophageal ultrasound. This is a tremendous improvement over current technology that requires catheters with electrical wires that are bulky, difficult to maneuver, and are consequently relatively large.

In this preferred embodiment of the device, the nanoparticle coated stent can be used either on DES, or BMS, with the PPTT effect being separate from the mechanical and chemical purposes of the stent. As such, the PPTT can be implemented at any time subsequent to implantation of the stent, or during the initial implant. The irradiation of the nanoparticles at their resonant frequency can be implemented either externally, with a strong NIR source placed at the surface of the tissue, or internally. Internal illumination can be provided either via a fiber-optic catheter inserted near the stent, or via any other internally placed excitation source. As an example of this, for a cardiac stent, the excitation source can be inserted within the esophagus, utilizing the penetration depth of the NIR or similar light source to penetrate to the implanted stent. Another method would be to insert a subcutaneous excitation source, inserted to within range of the penetration depth of the excitation wavelength in tissue.

This embodiment of the device is not intended to be limited to Cardiac stents, but also can be applied to any vascular stent. The differences lie in the size of the stent and the opportunities for applying the excitation. In embodiments where the stent is used in peripheral vascular systems, the external application of excitation electromagnetic radiation is preferred due to the relative proximity of the vessels to the skin, with completely non-invasive excitation possible using a strong energy source, or via a relatively non-invasive procedure. An example of an external skin application might be in the case of congenital structure narrowing, such as in infants with Congenital Heart Disease (CHD). In this case, the heart is very near to the skin, and the required penetration distances are small, thus allowing external excitation of a multi-use stent to ablate tissue as often as is necessary in small safe discrete increments.

In an alternative embodiment of this invention, the nanoparticles can be applied to the surface of a temporary carrier, such as a balloon used for intra-vascular dilating procedures. The energy source in this case might be situated on the catheter carrying the balloon. The energy source, such as an LED or laser, can thus be precisely directed to the area of interest on the balloon, thereby spatially controlling the area to be affected by PPTT. Sensors, such as temperature sensors can be placed immediately adjacent to the light source so that the therapeutic dose of energy can be precisely monitored and controlled. In this case, the therapist might choose to perform a conventional Angioplasty using the balloon only, followed by heat treatment to prevent subsequent vascular growth, or, the primary procedure can be the PPTT. This approach allows the ablation to occur even when a stent is not applied, or, it allows follow up ablation in cases where a previously placed BMS is starting to close, or even to treat a failed DES.

In this embodiment, the nanoparticles are stationary on the outer surface of the balloon, attached using methods of physical or chemical absorption, or are prefabricated on the vessel. The nanoparticles are positioned adjacent to the tissue surface via inflation of the balloon, and the PPTT can occur on the entire surface area of the balloon, or other vessel, using an isotropic energy source, or are directed onto specific regions of the vessel via the directional focusing of the energy source using optical methods. The directed focusing can be implemented using the fiber-optic within the catheter.

In other words, a balloon on a catheter, placed either in the lumen of the vessel, or placed at the position of exit where the vessel junctions with the chamber, so that the tip of the catheter is positioned by the vessel lumen and the body of the balloon, when expanded, contacts the chamber wall surrounding the vessel opening, will require minimal catheter positioning, will apply equal pressure to all contact points and will achieve superior contact with all possible target points. It is far more likely to “complete the circle” while requiring very little position adjustment.

Used in this way, the catheter is self centering and is self positioning. This eliminates the major usability limitations of current energy delivery catheter systems. A significant improvement is achieved even if current energy delivery methods, e.g., excessive heat, RF, or Cryo (cold), are still used, but are positioned by the balloon and by the self centering catheter. For example, an RF delivery system may be adjacent to the leading edge of the balloon, perfectly targeting the critical tissue. A Cryo fluid could then be passed via an appropriate lumen to the balloon tip for Cryo-Ablation.

Improved self centering could be achieved by using a two stage balloon. The most distal part of the balloon would enter the vessel and expand to center the catheter inside the lumen of the vessel. The more proximal part of the balloon, either immediately adjacent to the distal balloon or somewhat more proximal to it, would interface with the opening of the vessel into the chamber. Used in this way, nearly perfect centering of the therapeutic heat delivery balloon is achieved.

In addition, appropriate metallic particles, such as gold nanoparticles, may coat the leading edge of the heat delivery balloon. Heavy bulky electrical wiring would no longer be needed, which are part of existing high energy devices, yielding a thinner and more maneuverable catheter. Additionally, manufacturing costs would be drastically reduced, translating into lower costs for superior medical care. An energy source for optical wavelength delivery would still be required. However, this could be a light weight thin optical fiber or other optical source. The optical component could also be part of the catheter, or it could be a reusable or a single use component that is inserted, much as current catheter technology allows wires to be passed down the lumen of the catheter for various uses.

Therefore, the nanoparticle system and method according to the present invention would offer considerable advantage. The total energy output is a direct function of the particle size and characteristics, and the number of excited particles, thereby resulting in considerable control over energy actually delivered to tissue. Furthermore, the energy only needs to be delivered to tissue immediately adjacent to the particles. Precise delivery of the energy is thus assured. Alternatively, circumferential heating requires only that energy of the correct wavelength be directed evenly over the desired circumference.

The nanoparticle procedure according to the present invention is now simplified. The catheter, which is markedly more flexible and maneuverable, is positioned and self centered rapidly at the orifice of a vessel, the balloon is inflated to interface with the target tissue, and energy is applied in small increments until the desired result is attained. Should the therapy prove to be insufficient, the procedure may be repeated as often as needed to attain a permanent solution. Excessive tissue destruction and scaring is prevented, other complications are reduced or avoided, time is saved, and costs are significantly reduced.

Alternatively, used as a modified existing procedure, many of these benefits are still possible. Using the catheter's self-centering feature and with its balloon, the existing energy/Cryo mechanism, which is attached to or adjacent to the self-centering balloon according to the present invention, will perfectly interface with the target tissue. Alternatively, it may even be possible to deliver much smaller energy doses while still using these traditional energy sources due to the more precise catheter positioning and target tissue interface achieved by the present invention.

Such a targeted ablation technique may be used for vascular ablation, esophageal ablation, and others. Using a balloon-nanoparticle methodology, it should be possible to deliver more precise and lower energy, yet equally effective, or, possibly more effective energy for local tissue destruction. Complications, most significantly scaring, should be reduced. Further, due to the delivery by balloon, either conventional energy as a source, or nanoparticles as the medium of energy delivery, the ablation is both more focally precise and less likely to miss critical tissue. This is because current catheters are much smaller in diameter than the esophagus. Consequently, the catheters must be manipulated, often with direct visual guidance, using for example a fiber optic esophageal scope that is adjacent to the ablation catheter. The partially collapsed esophagus may obscure critical tissue, leading to it not being ablated. Also, the ablation catheter may obscure parts of the esophagus from the scope. However, when a balloon is employed, it will fully expand the esophagus, allowing either a fiber optic esophageal scope a clearer view, or, allowing visualization of the esophagus from a scope that is within an optically clear balloon.

Yet another disorder that can be treated using the targeted local heat ablation method according to the present invention includes asthma. The bronchial tree is extensive and lengthy in its totality. Selective, low energy treatments, applied to discrete areas could gradually increase function, one area at a time, without subjecting the patient to undue risk, and with a greatly reduced potential for severe injury. Additionally, dysplasia is a known precursor to cancerous degeneration in the bronchial tree in smokers, but also in others exposed to noxious stimuli. These dysplastic areas are seen with fiber optic brochoscopes. The balloon ablation technique according to the present invention could destroy this tissue just as described with respect to Barrett's Esophagus.

The targeted local heat ablation techniques of the present invention may also be used to treat sinus orifice narrowing, stenosis, or closure, narrowing of passageways in the GI tract, or narrowing of the genito-urinary tracts or passageways. Some examples of such genito-urinary tracts or passageways include passageways from the kidney to the bladder, including the critical junction areas, the passageway from the bladder via the urethra, including the bladder junction, the prostatic passage, and the urethra exit points in men, women, and children, are of which being possibly subject to disease induced, or to congenital narrowing. Women with diseases of the fallopian tubes, the passageways from the ovary to the uterus, may have either disease induced narrowing, or may be congenitally narrowed leading to reduced fertility or infertility. Women who have tube closure for birth control, and who later wish to reverse this decision, may have difficulty keeping their fallopian tubes open after surgical reversal. Women with excessive uterine bleeding may have ablation procedures to reduce the tissue thickness of their endometrial mucosa as a means of bleeding control.

All of these areas are examples of applications of the ablation techniques described using the balloon and nanoparticle gentle energy methods according to the present invention for which significantly improved results may be attained.

Yet another embodiment of this invention is similar to the previous embodiment, with the nanoparticles on the outer surface of a balloon or similar vessel. However, the vascular catheter may be replaced with an esophageal catheter to perform localized and well-controlled ablation procedures. This can replace the traditional Stretta procedure for the treatment of gastroesophageal reflux disease (GERD), where the ablation is induced via radio-frequency electrodes positioned on the outer edge of a balloon. The invention used for this purpose can replace the current Stretta procedure without the need for high voltage leads, and the resulting residual damage to the adjacent tissue. Other GI examples might include ablation or lesion removal in the esophagus, gastro-esophageal region, or, scar based local obstructions in the intestines or Gallbladder and its drainage system, the biliary tree. Additionally, other tubular structures, including the entire Genito-urinary systems, the sinus and auditory systems, pulmonary structures, and, in general, any area of the body that would benefit from controlled focal ablation.

The method of attaching the nanoparticles to the vessel or stent in the device includes pre-fabrication of nano-sized metallic structures; physically embedding nanoparticles and structures using physical deposition methods and imprinting; chemical absorption of the nanoparticles from solution phase; chemical attachment of the nanoparticles using covalent binding methods or other proteins and other electro-chemical deposition methods.

In all embodiments of this device, multiple types or sizes of resonant nanoparticles may be used within the device. This includes the use of nanoparticles of different size, composition (solid or core-shell structures) and materials. By varying the types of nanoparticles used, different regions of the device can be excited by different excitation wavelengths, this can allow further ablation control by selectively resonating only a certain fraction of the nanoparticles using only their resonant frequencies. In addition, if the nanoparticles are damaged or destroyed in an initial PPTT irradiation, secondary layers can allow additional PPTT treatments at later times, and at different wavelengths.

The above, and other aspects, features and advantages of the present invention, will become apparent from the following description read in conjunction with the accompanying drawings, in which like reference numerals designate the same elements, all of which form a part of this specification.

### BRIEF DESCRIPTION OF THE DRAWINGS

A further understanding of the present invention can be obtained by reference to a preferred embodiment set forth in the illustrations of the accompanying drawings. Although the illustrated preferred embodiment is merely exemplary of methods, structures and compositions for carrying out the present invention, both the organization and method of the invention, in general, together with further objectives and advantages thereof, may be more easily understood by reference to the drawings and the following description. The drawings are not intended to limit the scope of this invention, which is set forth with particularity in the claims as appended or as subsequently amended, but merely to clarify and exemplify the invention.

For a more complete understanding of the present invention, reference is now made to the following drawings in which:

FIG. 1 depicts different types of plasmonic and infrared excitable nanoparticles that may be used in accordance with the preferred embodiment of the invention, which are impinged upon with electromagnetic radiation and then emit thermal radiation.

FIG. 2 depicts a side view cross-section of a stent within a vessel that is being electromagnetically excited from outside the body in accordance with the invention.

FIG. 3 depicts a side view cross-section of a stent within a vessel that is being electromagnetically excited from within the body, by insertion below the outer skin layer in accordance with the invention.

FIG. 4 depicts a side view cross-section of a stent with a catheter light source threaded within the diameter of the vessel and stent that is illuminating the stent uniformly and isotropically in accordance with the invention.

FIG. 5 depicts a side view cross-section of a stent with a flexible, directional light source threaded within the diameter of the vessel and stent on top of a catheter, illuminating the stent directionally and locally, in accordance with the invention.

FIG. 6 depicts a side view cross-section of a catheter and balloon within a vessel, with the catheter illuminating the balloon uniformly and isotropically, in accordance with the invention.

FIG. 7 depicts a side view cross-section of a catheter having a directional light source on top and a balloon within a vessel, with the catheter illuminating the balloon directionally and locally, in accordance with the invention.

FIG. 8 depicts a side view cross-section of a balloon encompassing a first end of a tube having a directional light source within, and expanded along a vessel, with directional and local stimulation, mimicking the Stretta procedure in accordance with the invention.

FIG. 9 depicts the possibility of attaching multi-modal particles on a medical device, allowing multiple resonant wavelengths to excite and thermally heat the external walls of a vessel in accordance with the invention.

### DETAILED DESCRIPTION OF THE PREFERRED EMBODIMENTS

As required, a detailed illustrative embodiment of the present invention is disclosed herein. However, techniques, systems, compositions and operating structures in accordance with the present invention may be embodied in a wide variety of sizes, shapes, forms and modes, some of which may be quite different from those in the disclosed embodiment. Consequently, the specific structural and functional details disclosed herein are merely representative, yet in that regard, they are deemed to afford the best embodiment for purposes of disclosure and to provide a basis for the claims herein which define the scope of the present invention.

Reference will now be made in detail to several embodiments of the invention that are illustrated in the accompanying drawings. Wherever possible, same or similar reference numerals are used in the drawings and the description to refer to the same or like parts or steps. The drawings are in simplified form and are not to precise scale. For purposes of convenience and clarity only, directional terms, such as top, bottom, up, down, over, above, and below may be used with respect to the drawings.

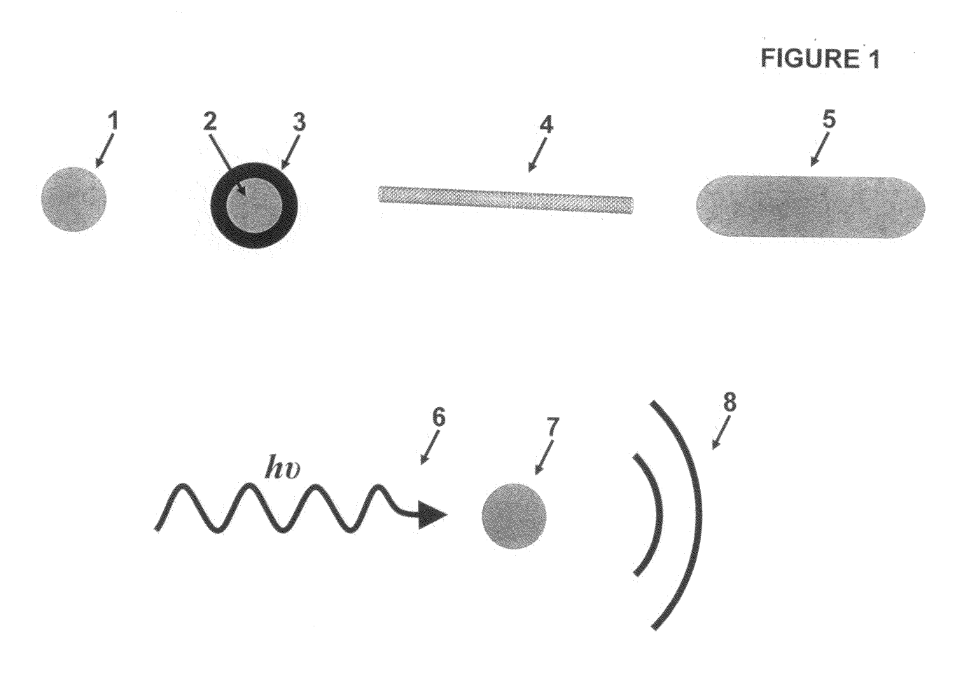

Turning first to FIG. 1, shown are different types of plasmonic and infrared excitable nanoparticles that may be used in accordance with the preferred embodiment of the invention. As depicted, the nanoparticles may consist of a variety of subtypes that are excitable by electromagnetic radiation. The simplest variety of these are the metallic, plasmonic nanosphere 1 which may be made out of any metal that exhibits plasmonic resonance in the visible and infrared regimes. Such metals include silver (Ag), gold (Au), platinum (Pt) and copper (Cu). Varying the size of these nanospheres, from a few to tens and hundreds of nanometers alters the resonance frequency of these structures (with a preferred size lying between 1-200 nm). A second subset of nanoparticles are core-shell structures that consist of a metallic shell 3 of varying thickness coating the internal, dielectric central core 2. Varying the thickness of the shell 3 alters the resonance frequency of the core-shell structure. A nanotube 4, typically consisting of the carbon nanotube variety, preferably having a diameter of 1 to 5 nanometers (nm), and lengths from tens of nanometers to several hundred microns (with a preferred length of less than 1 micron) is another type of nanoparticle that can be electromagnetically excited, typically with either infrared, or radio-frequency radiation. Altering the diameter and type of nanotube may alter the resonant frequency for infrared excitation, whereas radiofrequency excitation is less affected by structural alteration of the nanotube. Finally, an asymmetric nanorod 5 that has an ellongated axis providing one resonant frequency, and a shorter axis providing a second resonant frequency may be used in accordance with the present invention. Altering the aspect ratio and size of these nanorods (or nanowires) alters their respective resonant frequencies.

In the preferred embodiment of the present invention, electromagnetic radiation, consisting of impinging monochromatic, filtered, or even broadband frequencies 6, may be used to excite nanoparticles 7. The type of radiation used depends on the specific resonance, or resonance sets of the nanoparticle 7. When excited at resonance, the metallic nanoparticle 7 exhibits a plasmonic response due to the oscillation of the electrons in the metal, thereby causing excess heat buildup in the nanoparticle, which is then emitted as thermal radiation 8 that is local to the environment.

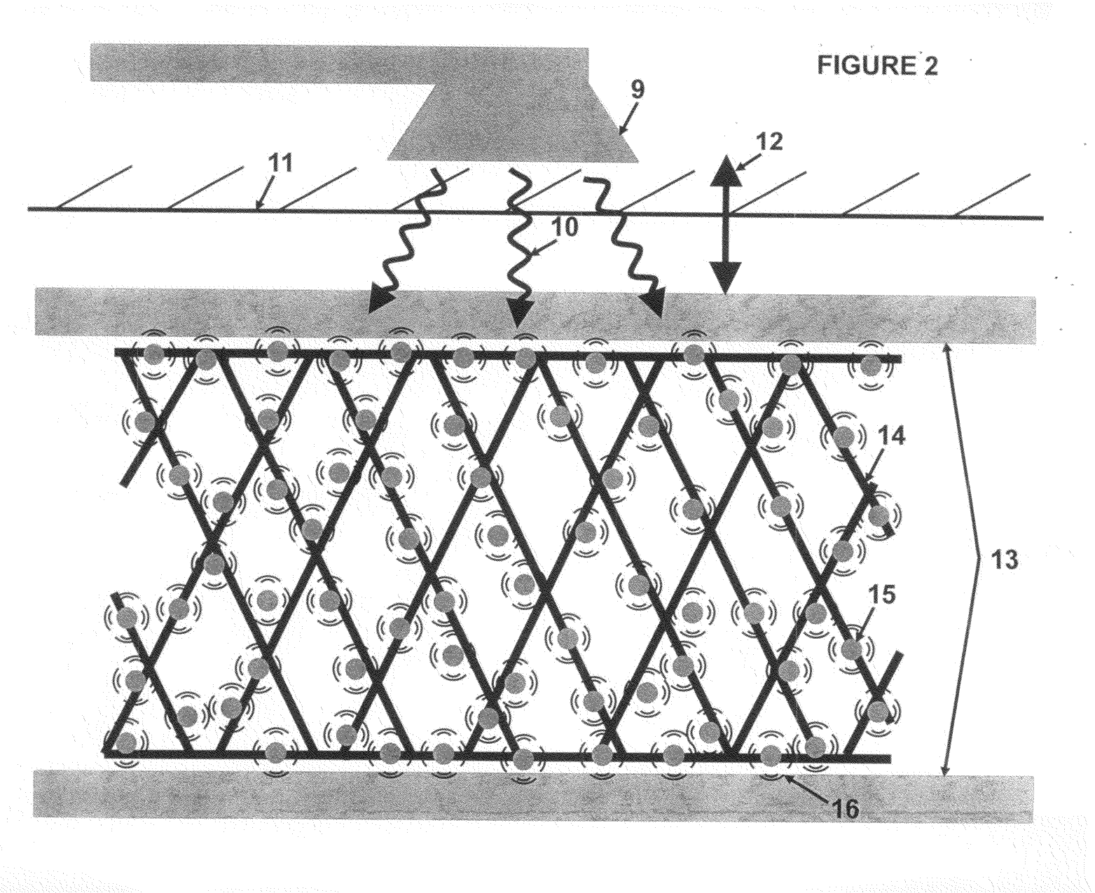

The preferred embodiment of the present invention is shown in FIG. 2. Here, shown is an external electromagnetic (EM) radiation source 9, which may include a lamp, laser, light emitting diode (LED), microwave or radio-frequency (RF) source (hereby defined as ‘source’) positioned external to the body 11 such that it is in a non-invasive manner. The device in accord with the invention is preferably used in conjunction with a nanoparticle coated stent 14 lying within an internal section of the body, for example within a blood vessel 13. Since EM source 9 is external to the body, the distance between EM source 9 and stent 14 may be quite large 12 (e.g., in the range of a few millimeters to several centimeters). This range is a limiting factor of the device (e.g., the stent and nanoparticles), and is dependent upon the wavelength 10 used to excite nanoparticles 15, due to the low penetration depth of most visible light within tissue. The nanoparticles in this embodiment are illuminated isotropically, with no selectivity, such that all the nanoparticles 15 are equally excited, which then emit thermal radiation thereby thermally heat the vessel walls 13 adjacent to the device to cause ablation of the targeted tissue. This PPTT process is a local reaction, causing only cellular material of the tissue in the direct vicinity of the excited nanoparticles to be ablated due to the thermal resonance response 16.

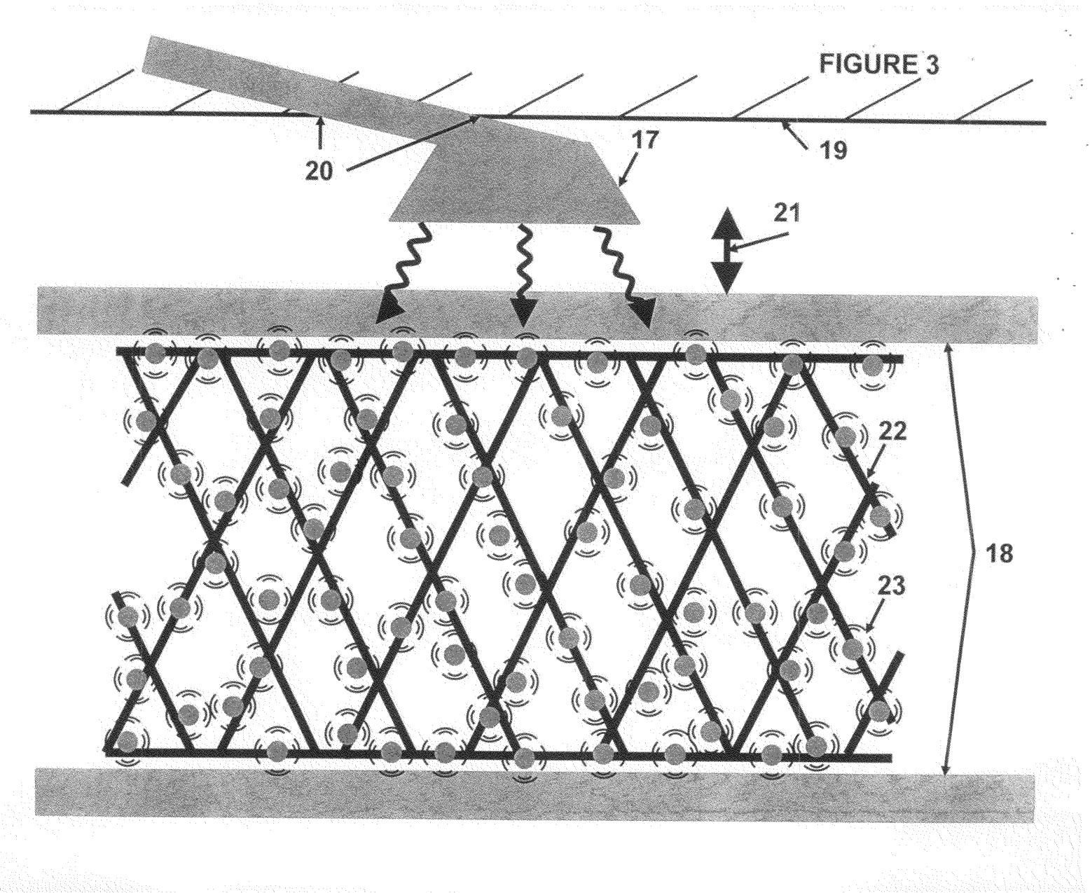

An alternative embodiment is shown in FIG. 3, whereby EM source 17 is external to the vessel 18, but is within the layer of the outer tissue 19 (i.e., inside the body but outside the vessel). This embodiment requires a relatively benign invasive process of inserting the source 17 through the outer tissue (e.g., the skin) in a small puncture 20 such that the relative distance between the source and device 21 is lessened. By lessening the distance 21 between source 17 and stent 22, the penetration depth of the radiation is improved, allowing better excitation of the nanoparticles on stent 22. In this iteration, the excitation is isotropic and uniform over the length of the stent 22 such that all the nanoparticles 23 are equally excited, thereby inducing the PPTT.

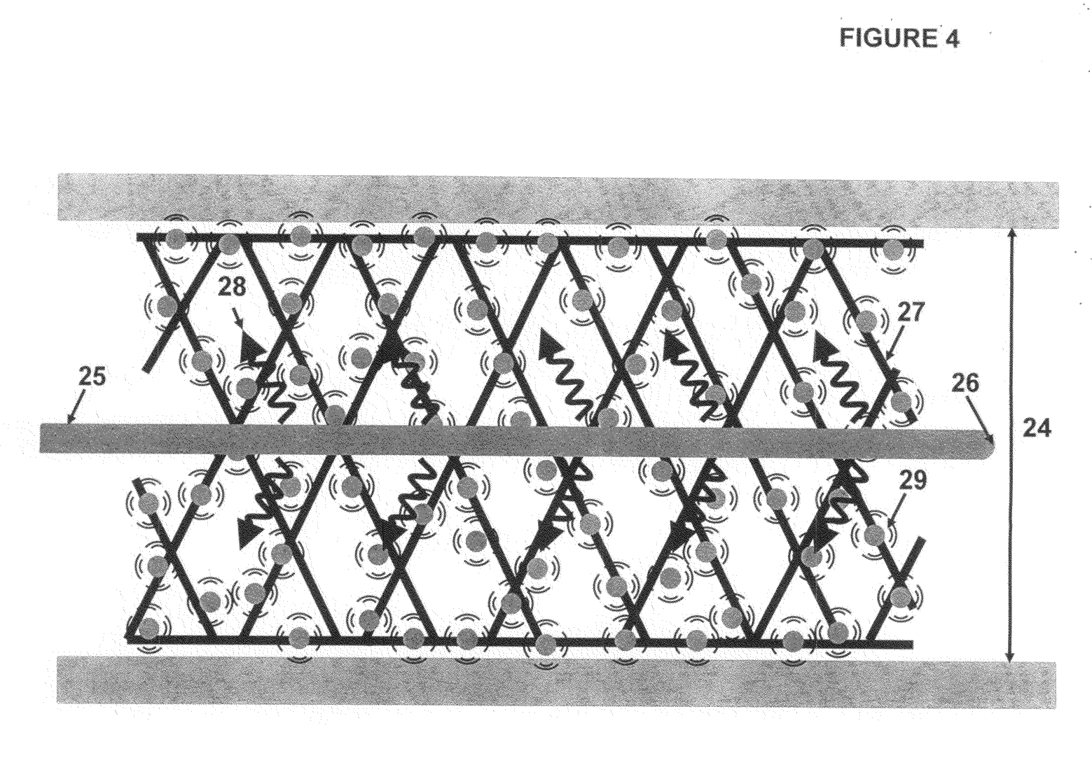

Still another alternative embodiment of the invention is shown in FIG. 4. Shown is catheter 25 inserted in an existing stent 27 lying within a vessel 24. An example of when this can occur is during the insertion of the stent itself, or at a later stage, when thrombosis is detected. The catheter 25 may be inserted such that an end 26 of an optical fiber (e.g., via catheter 25) reaches the full length of the stent 27 and illuminates 28 the stent 27 from within in an isotropic and uniform manner, thus exciting the nanoparticles 29 on the stent 27.

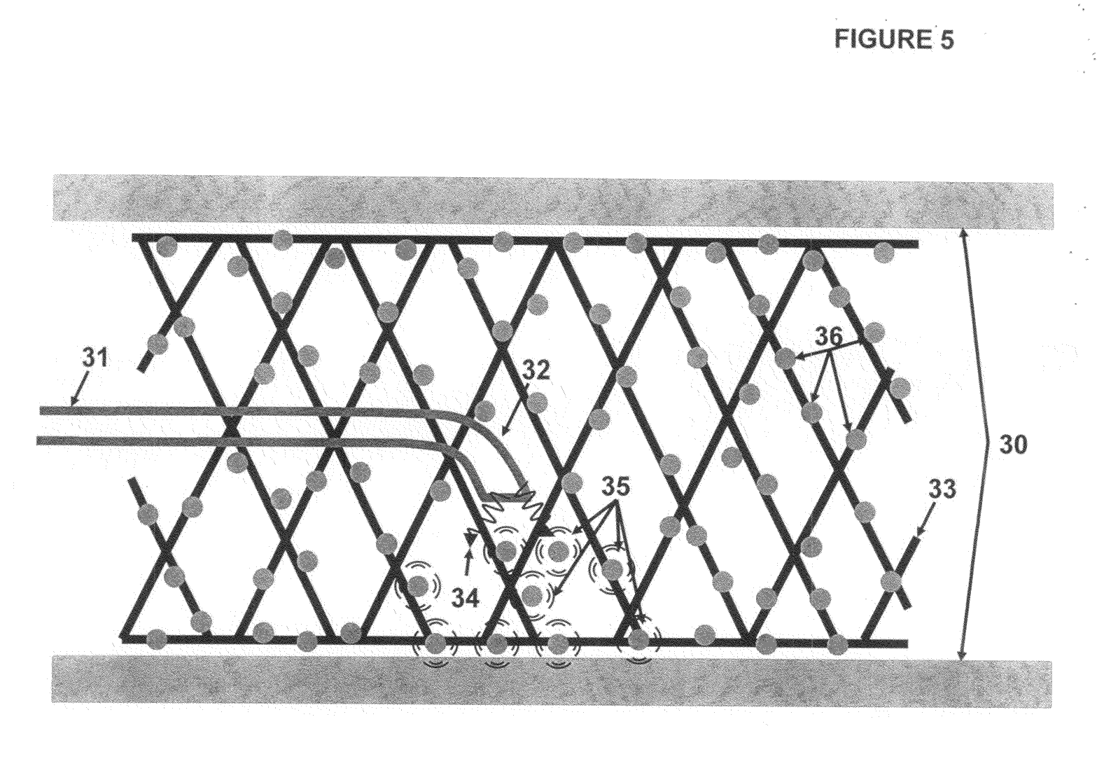

Another embodiment of the device in accordance with the invention is shown in FIG. 5. As shown, stent 33 lies within a vessel 30, and a optical source on a catheter 31 is inserted into the stent 33, with a directional head 32. In this embodiment, the EM excitation 34 emitted from the head 32 is directional, such that only nanoparticles 35 in a local section of the stent 33 are excited. The remainder of the nanoparticles 36 on stent 33 remain inert. In this embodiment, the catheter 31 may be moved along the length of the stent 33 and thereby create a PPTT response in specific desired local regions of the stent 33. In this embodiment, the entire stent 33 can be excited via the source in or on catheter 31, or only a local region of the stent 33 can be excited, thereby limiting the ablation damage to healthy regions of the vessel 30.

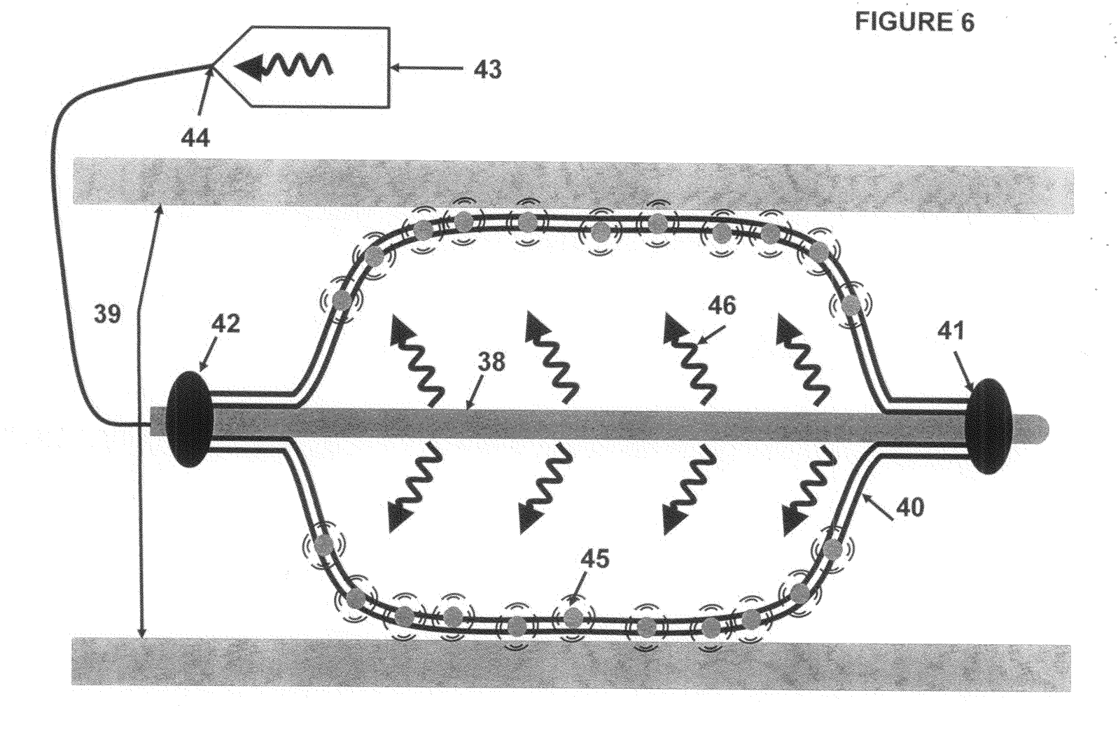

Still another embodiment of this device of the invention is shown in FIG. 6. Here, shown is an inflatable balloon 37 that is placed on a catheter 38 to inflate a vessel 39. Preferably, the catheter 38 is placed in the required, damaged region such that the balloon 40 is lined up with the desired section. The catheter 38/balloon 40 device is inflated from both ends 41, 42 of the catheter 38, thereby distending the balloon 40 the required amount of distance to touch the walls of vessel 39. The catheter 38 is preferably fitted with an external source 43 that is coupled via optical fiber 44 into the catheter 38 that uniformly and isotropically illuminates the nanoparticles 45 with EM radiation 46 on the outer edges of the balloon 40. These nanoparticles 45 on the external section of the balloon 40 will thereby induce the PPTT.

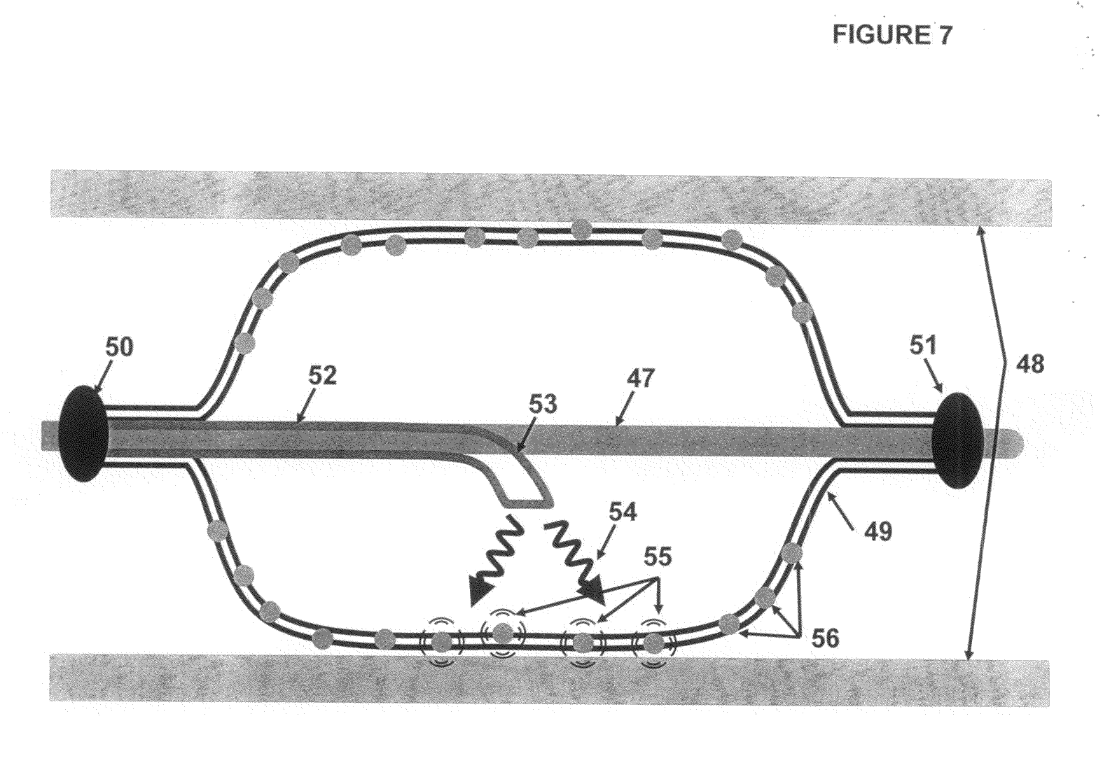

In a similar embodiment to that shown in FIG. 6 shown in FIG. 7 is catheter 47 placed within a vessel 48 to the desired location, and the balloon 49 is inflated to the desired volume. The balloon 49 is held in place by seals 50, 51 at both ends of balloon 49. In this embodiment, an EM radiation source 52 having a directional head 52 is placed on catheter 47 such that directional head 52 may illuminate and excite nanoparticles 55 in a spatial and local fashion as indicated at 54. Only those nanoparticles 55 directly illuminated with energy 54 by the directional head source 50 are excited, and the remainder of nanoparticles 56 remain inert. In this embodiment, the entire balloon 49 can be excited via the catheter source 52, or only a local region of the balloon 55 can be excited, thereby limiting the ablation damage to healthy regions of the vessel.

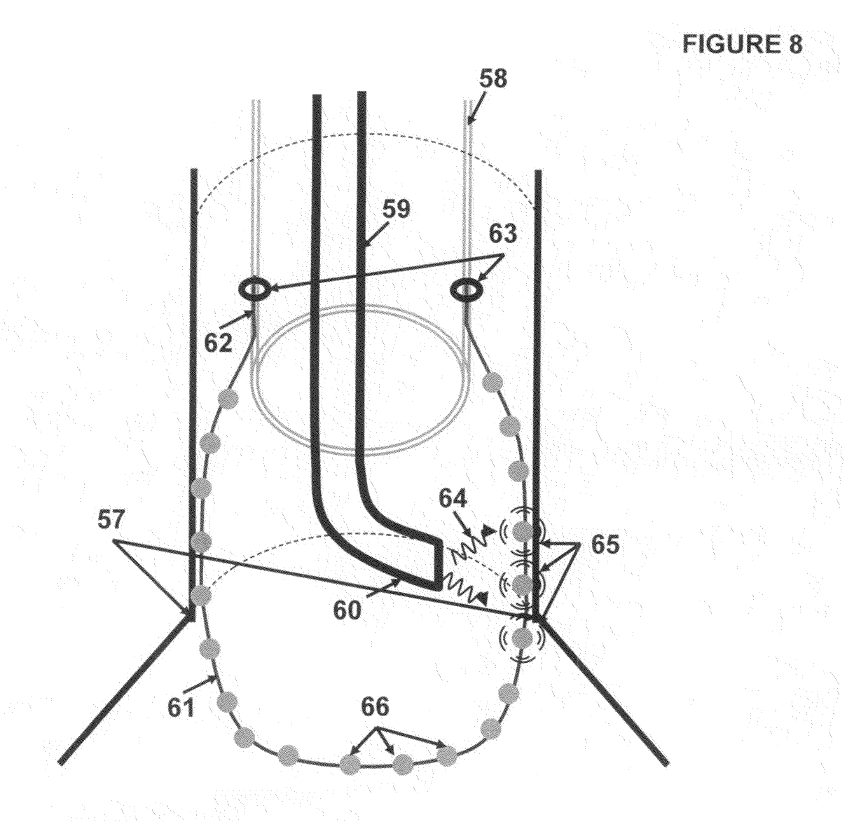

In another embodiment of this device, shown in FIG. 8, a procedure similar to the Stretta procedure for the treatment of GERD is shown. Here, the gastro-esophageal junction 57 is slightly inflated using balloon 61 placed on tube 58 and inserted within the esophagus. The balloon 61 is attached to an open end 62 of tube 58 and held in place by a ring 63. The balloon 61 is preferably coated externally with nanoparticles 66. A catheter or similar device 59 is inserted within tube 58 preferably with a directional head source 60. This source 60 locally and spatially illuminates with energy 64 specific regions of the balloon 61, thereby exciting only specific nanoparticles 65, leaving the remainder of the nanoparticles 66 inert. In this embodiment, the directional source 60 may be rotated and aligned to create patterns of ablation regions along the surface area of the balloon 61. In this embodiment, there is no need to uniformly excite the nanoparticles on the balloon 61, however, this can be done as well if desired. Alternatively, for atrial fibrillation ablation the target area is typically the forward part of the balloon, rather than at or near the center circumference of the balloon. In this situation, the tip of the catheter is preferably used to locate and hold the catheter in position. Importantly, since most ablations are done in a Cath lab under fluoroscopic guidance, the openings to the pulmonary veins may not be seen. For larger vessels, however, the balloon may be elongated, or a second smaller balloon may be positioned forward to the primary balloon, which will hold the catheter in a substantially centered position.

In accordance with the present invention, the nanoparticles may be embedded onto the devices described, being either stents, catheters, balloons, or other devices, using any number of techniques, including physical embedding, chemical binding, or electroplating.

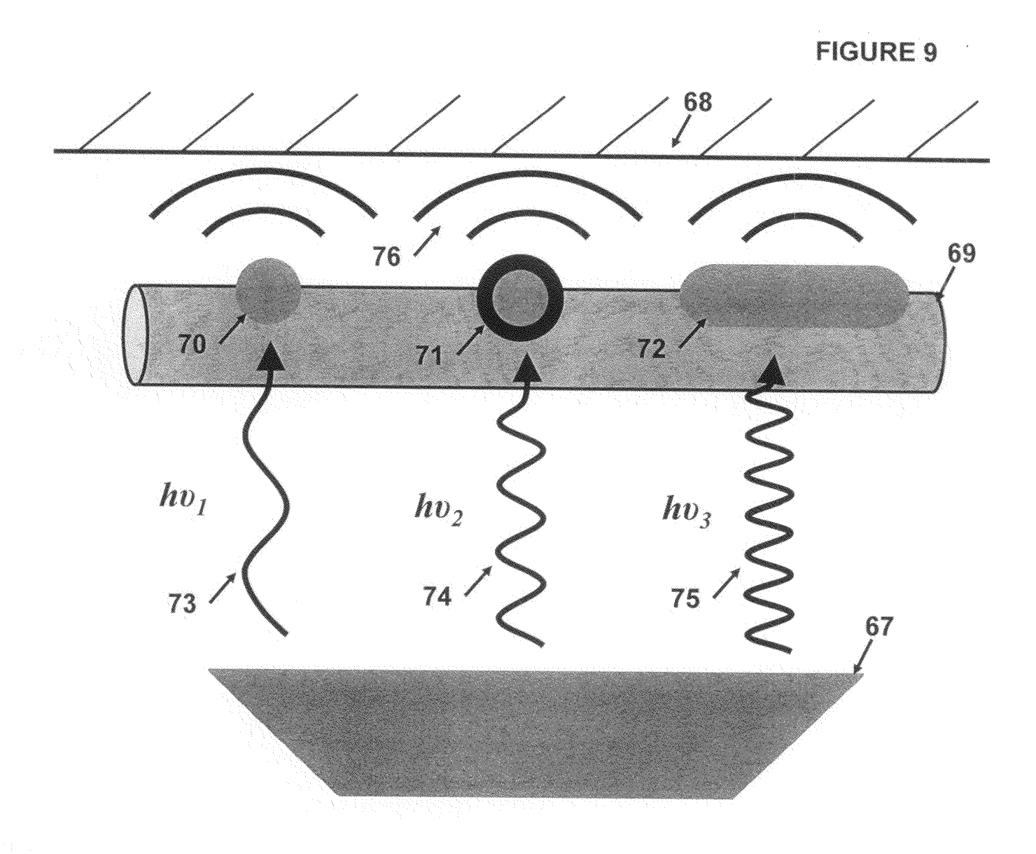

In all embodiments of the invention, more than a single type of nanoparticle may be used on the same device. For example, as shown in FIG. 9, multiple different types of nanoparticles (e.g., nanosphere 70, core-shell nanosphere 71, or nanorod 72) are embedded onto a surface of device 69. In this type of device, a target tissue that is to be ablated 68 and a device 69 are in close proximity A source 67, being external (not shown) or internal (shown), is used to excite the nanoparticles on the device. In the schematic of FIG. 9, three different types of nanoparticles are shown, nanospheres 70, core-shell nanostructures 71, and nanorods 69. Preferably, each of these nanoparticles has its own, distinct resonance frequency 73, 74, 75 at which they are excited and produce a PPTT response 76. For example, nanosphere 70 may have a resonant frequency hv 1 73, core-shell nanosphere 71 may have a resonant frequency hv2 74, and nanorod 72 may have a resonant frequency hv3 75. In this multimodal response, the nanoparticles can optionally be of the same subset, but of different resonance frequencies. For example, only nanospheres 70 may be used having different diameters or nanorods 73 may be used having different aspect ratios. Each nanoparticle is then excited only when the appropriate resonance frequency is used, leaving the other nanoparticles inert. In this type of multi-modal embodiment, different nanoparticles may optionally be placed at different segments of the device (e.g., stent, ballon, etc.), thereby creating spatial functionality even when uniformly excited by a source 67. In addition, different nanoparticles may optionally be excited at different times by illuminating them with the appropriate wavelength at different times, which could include separate procedures. This may be used in the event that some of the nanoparticles are destroyed or rendered inert in some other way, thereby extending the number of times the PPTT may be implemented.

In the claims, means or step-plus-function clauses are intended to cover the structures described or suggested herein as performing the recited function and not only structural equivalents but also equivalent structures. Thus, for example, although a nail, a screw, and a bolt may not be structural equivalents in that a nail relies on friction between a wooden part and a cylindrical surface, a screw's helical surface positively engages the wooden part, and a bolt's head and nut compress opposite sides of a wooden part, in the environment of fastening wooden parts, a nail, a screw, and a bolt may be readily understood by those skilled in the art as equivalent structures.

Having described at least one of the preferred embodiments of the present invention with reference to the accompanying drawings, it is to be understood that such embodiments are merely exemplary and that the invention is not limited to those precise embodiments, and that various changes, modifications, and adaptations may be effected therein by one skilled in the art without departing from the scope or spirit of the invention as defined in the appended claims. The scope of the invention, therefore, shall be defined solely by the following claims. Further, it will be apparent to those of skill in the art that numerous changes may be made in such details without departing from the spirit and the principles of the invention. It should be appreciated that the present invention is capable of being embodied in other forms without departing from its essential characteristics.

What is claimed is:

1. A method of locally heating tissue with the body using nanoparticles attached to a device and excited using an electromagnetic source, said method comprising the steps of:
attaching one or more nanoparticles to a device to be inserted into a body; inserting the device into the body; and
exciting the nanoparticles on the device using a light source.

2. The method of claim 1, wherein said nanoparticles have diameters within the range of 1 to 1000 nanometers (nm).

3. The method of claim 1, wherein the nanoparticles are metallic.

4. The method of claim 1, wherein the nanoparticles are metallic shells on non-metallic cores or core-shell structures.

5. The method of claim 1, wherein the nanoparticles are fabricated nanostructures.

6. The method of claim 1, wherein the nanoparticles are produced in solution.

7. The method of claim 1, wherein the light source is in the 380-2000 nm spectral range.

8. The method of claim 1, wherein the light source is tuned to the plasmonic resonance frequency of the nanoparticles.

9. The method of claim 1, wherein the light source is selected from the group consisting of a filtered lamp, a light emitting diode (LED), and a laser.

10. The method of claim 1, wherein the light source is infrared to radiofrequency.

11. The method of claim 1, wherein the nanoparticles are magnetic.

12. The method of claim 1, wherein the nanoparticles are spherical.

13. The method of claim 1, wherein the nanoparticles are of a shape selected from the group consisting of non-spherical, asymmetric spheres, cubes, pyramids and octahedrons.

14. The method of claim 1, wherein the nanoparticles are attached using a method selected from the group consisting of physical deposition techniques, chemical deposition techniques, physical absorption techniques, electro-chemical techniques, and covalent binding techniques.

15. The method of claim 1, wherein the device is selected from the group consisting of a bare metal stent, a drug eluting stent, a vessel on a catheter, and a vessel on an esophageal catheter.

16. The method of claim 15, wherein the vessel is selected from the group consisting of a balloon, and an inflatable polymer.

17. The method of claim 1, wherein exciting the nanoparticles heats the targeted tissue.

18. The method of claim 1, wherein multimodal excitation is used to excite different resonances in different types of nanoparticles.

19. The method of claim 18, wherein the nanoparticles are of the same type, each having different resonances due to their geometry.

20. The method of claim 18, wherein the nanoparticles are of different types, each having different resonant frequencies.

21. The method of claim 18, wherein the nanoparticles are spatially separated on the device.

22. The method of claim 21, wherein said spatial separation of said nanoparticles allows spatial control of ablation.

23. The method of claim 18, wherein the nanoparticles are uniformly distributed.

24. The method of claim 1, wherein the nanoparticles are illuminated at different frequencies at different times.
